(19) United States
(12) Patent Application Publication
(10) Pub. No.: US 2018/0160904 A1
(43) Pub. Date: Jun. 14, 2018

(54) REHAB READY APPLICATION

(71) Applicant: Alejandro Saenz, Arlington, TX (US)

(72) Inventor: Alejandro Saenz, Arlington, TX (US)

(21) Appl. No.: 15/842,558

(22) Filed: Dec. 14, 2017

Related U.S. Application Data
(60) Provisional application No. 62/433,863, filed on Dec. 14, 2016.

(51) Int. Cl. A61B 5/00; A63B 24/00; A61B 5/11; A63B 71/06

(52) U.S. Cl. CPC A61B 5/0002; A63B 24/0062; A61B 2505/09; A61B 2503/10; A63B 71/0622; A61B 5/11

(57) ABSTRACT

Rehab Ready Application assists users in their efforts to diagnose injury and suggest treatments through audiovisual recordings, photographs, written or oral instructions for treatment, and teleconferences or referrals to medical providers. Users indicate location of pain, mechanism of injury, level of pain, type of pain, degree of pain, range of movement and other symptoms. The application recommends specific exercises, repetitions, and duration for the treatment. Users can choose to view photos or recorded demonstrations of the treatment, written instructions, or a teleconference with a medical professional who can demonstrate the treatment and/or observe the user perform the treatment and offer feedback. Depending on the user's progress, additional treatment may be recommended or the user may be scheduled for an in-office visit with a medical professional if the injury is too severe for treatment via the application. Rehabilitated users may receive a recommendation for future exercise and treatment to prevent re-injury.

The present disclosure relates generally to an applicators for the diagnosis of physical ailments and virtual treatment of a patient by a medical professional.

### BACKGROUND

People who engage in sports, exercise and other athletic endeavors are oftentimes injured during participation. Some athletes ignore the injury without medical intervention due to time or money constraints. Some injured parties may even choose to self-diagnose and self-treat based on their own research. For those athletes with access to insurance or money for treatment, they may pursue diagnosis and treatment from a physical therapist, chiropractor, or a medical doctor. However, making an appointment and taking time away from work or family to attend the appointments are additional obstacles to treatment and recovery. Once the injury is diagnosed and a treatment plan is given to the patient, the patient will have to attend physical therapy for weeks or months in an effort to recover from the injury. Some people may not make the time in their daily schedule to properly rehabilitate the injured body part due to the ongoing inconvenience and time required to seek out treatment.

Systems have recently been created to assist physical therapists and other medical professionals with the diagnosis and treatment of athletic injuries. These applications (“apps”) and websites cater to those with medical training, the ability to understand the technical verbiage, and a base level of familiarity with common treatment exercises. These types of systems are inappropriate for civilian use since they are likely too complex for a regular person without medical training. Those systems designed for use by common people are not specifically tailored to an individual but are directed towards a group of people who have self-diagnosed their injury. For instance, if a user believes they have an issue with an ankle, calf or shoulder, the user would then search treatment options for that particular body part. However, if the pain is secondary to the actual injury, the user would not find a treatment plan or video to treat the injury if the user has initially misdiagnosed his/her injury.

Current applications do not allow the user to assess/diagnose their injury and then find the best treatment for their injury, whether nerve restriction, muscular strains, tendon/ligament damage, or bone damage. Most applications provide general rehabilitation or information on different types of injuries. Currently available apps do not help in the assessment process or diagnosis process of an injury but rather list possible injuries and possible treatment. This information is not helpful for the average person without medical training to properly assess and diagnose an injury.

Currently available apps are designed for medical professionals who understand the medical terminology used and therefore may not be helpful to laypersons. Apps designed for the common user, without medical training or expertise, do not provide a comprehensive assessment and understanding of the injury and therefore are not capable of providing an appropriate treatment plan. The present application will bridge this gap by integrating professional medical information into a structured stepwise format that any common user can navigate.

Recreational athletes or weekend warriors may sustain injuries, whether a sprain, strain, or twist) as frequently as every month. It's not practical or financially sound to seek medical attention through an office visit or consultation for each occurrence of what appears to be a minor injury that may be treated and healed with exercise or stretches. Hence, many recreational athletes and laypersons will see out medical guidance from the internet (i.e. WebMD, Mayo Clinic, etc.) based on their symptoms. The user is quickly overwhelmed with information, most of it irrelevant for the user's actual injury.

Similar products often provide a list of practically every type of injury or diagnosis, which frequently results in the user overanalyzing or overthinking their injury. In turn, the user may then ignore the injury and work through the pain or seek out medical attention for an issue that does not require medical intervention to property treat and heal. Once seen by a medical professional, the user may receive a prescription for medication instead of pursuing a non-pharmaceutical option for recovery, such as exercise or physical rehabilitation of the injured body party. Once the medication has been consumed, the underlying injury and issue likely remain and may continue to cause issues for the user as the injury is aggravated again during daily use. Once a user is presented with a list of symptoms and possible ailments, the user is often subconsciously influenced towards a particular diagnosis and may become overwhelmed with the information.

Once a user enters the app, they will receive step by step guidance to determine the likely type of injury at issue. The app eliminates guesswork for the user by asking targeted, multiple choice questions, which will lead to an accurate determination of the injury based on differential medical analysis of the symptoms. If the user's inputs are consistent with a more serious injury, inappropriate for mobile diagnosis and treatment, the user will be referred to a network of approved medical professionals comprised of chiropractors, physical therapists, and medical doctors. The present disclosure will save user's hours of time spent searching the internet reviewing medical texts or navigating multiple apps/databases to determine how serious their injury is and appropriate treatments. The step by step process of the present application will allow a user to quickly eliminate certain injuries and zero in on the specific ailment currently distressing the user.

The present disclosure will assist users in their efforts to filter out the irrelevant and hone in on specific information relevant to their particular injury and treatment plan without the hassle of suffering from information overload, sifting through thousands of search results, suffering hypochondriac episodes after reading volumes of medical information and personal accounts and experiences. The present disclosure bridges the gap between information overload and in-office treatment from a medical professional. Users of this application can filter the information available, online and offline, in order to efficiently and practically determine and implement their diagnosis and treatment. However, if at any point a diagnosis cannot be made the app user will have the ability to either set an appointment with a local provider suggested by the app and affiliated network or the user will have the ability to have participate in a telemedicine consultation via video services such as Skype or Facetime. If a user enters information which indicates an emergency medical situation (i.e. tear, break, fracture, or any other severe injury), the user will be immediately referred to the emergency room, their primary care physician or other medical professional for immediate treatment of the injury or condition.

### OBJECTIVES

An object of one embodiment of the present disclosure is to provide a user with mobile access to a medical professional for the diagnosis and treatment of athletic injuries.

Another object of the present disclosure is the ability of a user to video record their injury/ailment and have it sent in to a network of providers in order to have a proper diagnosis if the injury does not fall into one of the app suggested diagnosed categories.

Another object of the present disclosure is to provide diagnostic exercises/tests for a user to perform to assist the medical professional with the diagnosis of the user's injury.

Another object of the present disclosure is to provide visual demonstrations and audio descriptions of the treatment plan for a user based on the diagnosed injury.

Another object of the present disclosure is to provide a referral network for users who require physical contact and treatment from a medical professional.

Another object of the present disclosure is to provide fast access to a library of rehabilitation exercises that can be used to heal an existing injury.

Another object of the present disclosure is to eliminate most of the uncertainty associated with self-diagnosing injuries, by providing a series of questions to determine the user injury.

Another object of the present disclosure is to provide an app appropriate for use by a range of users, from professional athletes to recreational sport participants to office workers.

Another object of the present disclosure is to provide a pre-treatment or pre-rehabilitation for possible injuries that may arise due to the nature of the activity.

Another object of the present disclosure is to provide a post-treatment rehabilitation program after the injury is resolved to prevent future re-injury of the aforementioned body part.

Another object of the present disclosure is to file an extensive history of each user which will include injury history, rehabilitation protocol, and medical professionals that have assisted them.

Another object of the present disclosure is to provide instant video feedback and interaction with in-network approved medical professionals via videoconference, teleconference, skype, facetime or other similar systems.

Another object of the present disclosure is that all medical providers will have to be pre-qualified in order to be a part of our network, which will include but not be limited to their profession, their own exercise experience/knowledge, involvement in our network and philosophy towards exercise and injury.

### BRIEF DESCRIPTION OF THE DRAWINGS

For a more complete understanding of this disclosure and its features, reference is now made to the following description, taken in conjunction with the accompanying drawings, in which:

FIG. 1 shows the RRA Injury Flow Chart and includes the following: Injured User, Mechanism of Injury, Specific Body Party, Pain Level, Type of Pain, and Injury Type.

FIG. 2 shows the RRA Injury Flow Chart and includes the following: Movement Screening to Determine Type of Injury, Assignment of Corrective Exercises, Audiovisual tutorials or Online consultations, User performance of exercises, User Progress Report, User release from treatment plan or Referral In-Network Medical Professional Advanced Strengthening Program to prevent re-injury.

FIG. 3 shows an alternative injury flowchart and diagnosis/treatment and includes the following: Body Part Area of Pain, Mechanism of Injury, Pain Type, Possible Injuries, Physical Tests, and Treatment Exercises; Payment information, Pain Remedy Protocol and Referral to Specialist for further treatment.

FIG. 4 shows the sample flowchart for diagnosis and treatment of an ankle injury.

FIG. 5 shows the sample flowchart for diagnosis and treatment of a wrist injury.

### FIGURE REFERENCES

1) Body Part/Area of Pain

2) Mechanism of Injury

3) Type of Pain

4) Pain Level

5) Possible Injury

6) Movement Screening

7) Treatment Option/Corrective Exercise/Treatment Plan

8) Medical Consult

9) User Self-Treatment/Exercise Program

10) Reassessment of Rehabilitation of Injury

11) Preventative advise

12) Referral to emergency room, primary care, or physical therapist

### DETAILED DESCRIPTION

The present disclosure generally provides an application for the RRA.

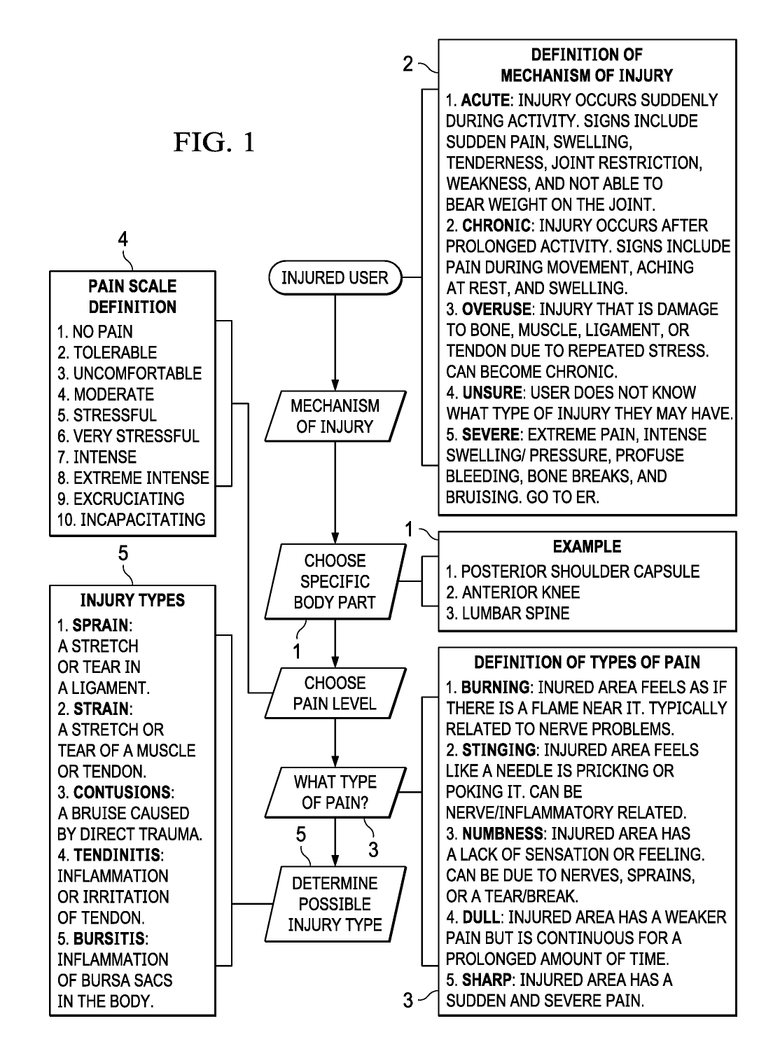

FIG. 1 shows an injury flowchart and diagnosis/treatment with RRA. The user will log into the RRA and select a “Mechanism of Injury” 2 which can be one of the following: Acute when the injury occurs suddenly during activity, Chronic when the injury occurs after prolonged activity. Overuse when the injury involves damage to bone, muscle, ligament, or tendon due to repeated stress. Unsure when the user is not aware of the type or cause of the injury, and Severe when the pain is extreme including intense swelling/pressure, profuse bleeding, broken bones and bruising at which time the app will advise this user to proceed directly to an emergency room. After the mechanism of injury is identified, the user will “Choose Specific Body Part” 1 such as a posterior shoulder capsule, anterior knee or lumbar spine and a level of pain on the “Pain Scale” 4 ranging from 1 (minimal/no pain) to 10 (intolerable pain). For example, the pain scale may be as follows:

1. Minimal/No Pain: Pain is present, but is not very noticeable. All activities and movements are unhindered. A more annoying type of pain like a bug bite.

2. Tolerable: Pain is present, and more noticeable. Minor pain that does not inhibit activities or movements. For instance, stubbing your toe on a table.

3. Uncomfortable: A stronger pain that is noticeable and cannot easily be ignored. Still does not inhibit activities or movements. Early tendonitis can fall under this.

4. Moderate: A strong pain that does not go away and begins to interfere with movement and activities. Does not require medication or assistance. A minor back sprain is an example.

5. Stressful: A deeper pain that interferes with activity and may require external management in order to function normally (i.e. ibuprofen or cold compresses). Ignoring the pain has become difficult but is still possible. An example is a herniated disc.

6. Very Stressful: A pain that requires may assistance to move around such as crutches or a brace. The pain has become great enough to cloud your thinking. Ignoring the pain is more difficult. An example is a high ankle sprain.

7. Intense: The pain mostly cannot be ignored and you think about the pain most of the time when you are awake. Normal activities can be performed with assistance but will cause pain. An example is a partially torn ACL.

8. Extremely Intense: The pain is very strong and needs over the counter pain medication or assistance in order to function somewhat normally. Is severely inhibited to participate in normal activities, but can perform some but with extreme pain. The ability to block this pain out is impossible. An example is a minor fracture of the tibia.

9. Excruciating: Pain that is so great, one cannot move or function without assistance or the use of very strong painkillers. Cannot engage in normal activities. An example is a broken ankle.

10.Incapacitating: Pain that causes one to pass out. Occurs due to extremely painful injuries such as crushed limbs. Not tolerable in ANY way. Need immediate relief. An example is a crushed femur.

Next, the user will identify the type of pain 3 the user is suffering from, which may include the following: Burning when the injured area feels as if there is a flame near it (i.e. nerve problems), Stinging when the injured area feels like a needle is pricing or poking the body party (i.e. nerve or inflammatory issue), Numbness when the injured area lack sensation or feeling (i.e. nerves, sprains or tear/break). Dull when the injured area has a weaker pain but is continuous for a prolonged period of time, and Sharp when the injured area has sudden and severe pain.

In order to diagnose the “Injury Type” 5, the user will select one of the following in the app:

Sprain, which is a stretch or tear in a ligament, Strain, which is a stretch or tear of a muscle or tendon, Contusion, which is a bruise caused by direct trauma, Tendinitis, which is inflammation or irritation of a tendon, and Bursitis, which is inflammation of bursa sacs in the body.

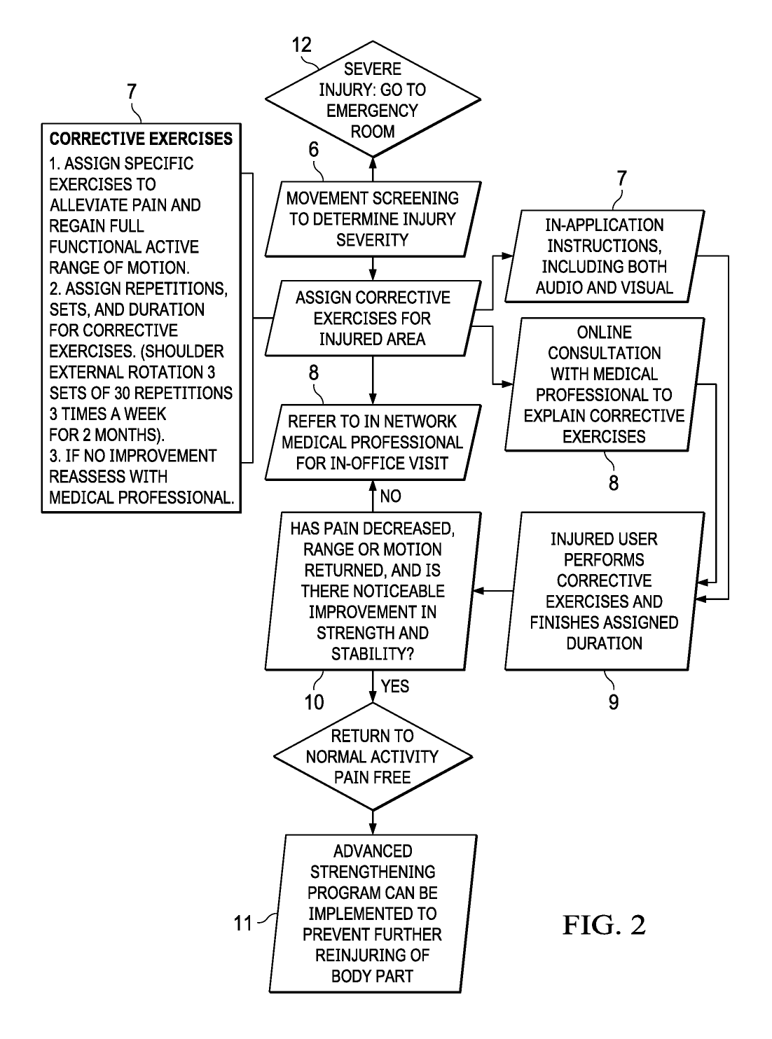

FIG. 2 shows an injury flowchart and diagnosis/treatment with RRA. After these initial determinations are made, the user will participate in “Movement Screening to Determine Injury Severity” 6 by performing a series of movements related to the specific body part and the injury/pain type. The results of the movement screening are input into the RRA in order to determine a proper course of treatment using video, pictures or written descriptions. If the app determines that the injury is too critical and/or severe for mobile treatment 12, the patient will be directed to go immediately to the emergency room since those injuries are beyond the scope of treatment of a mobile app.

If the app determines that the injury is not too severe for mobile treatment, the app will assign corrective exercises 8 to the user based on the specific body part 1 and the injury/pain type 3,5. Specific exercises 7 are chosen to alleviate pain and regain full, functional active range of motion. The app will assign the user a series of repetitions, sets and the duration for the specific corrective exercises (example: shoulder rotation, 3 sets of 30 repetitions, 3 days a week for 8 weeks). The user may either view pre-recorded instructional audiovisual videos, photographs of exercises, or written or audio descriptions of the exercises or treatments inside RRA or the user may participate in an online consultation with a medical professional (i.e. video and/or audio communication) to receive additional information on the prescribed corrective exercises. The user is then responsible for following the course of treatment for the suggested duration 9. Afterward, the user will input data into the app to indicate if the pains, range of motion, strength, and stability have increased, decreased, or not changed 10. If there has been no improvement or a worsening of symptoms, the user will be referred to an in-network medical professional for an in-office visit through RRA 8. If there has been significant improvement, the user may return to normal activity without pain. The user may also request an advanced strengthening program to prevent future re-injury of the body part 11.

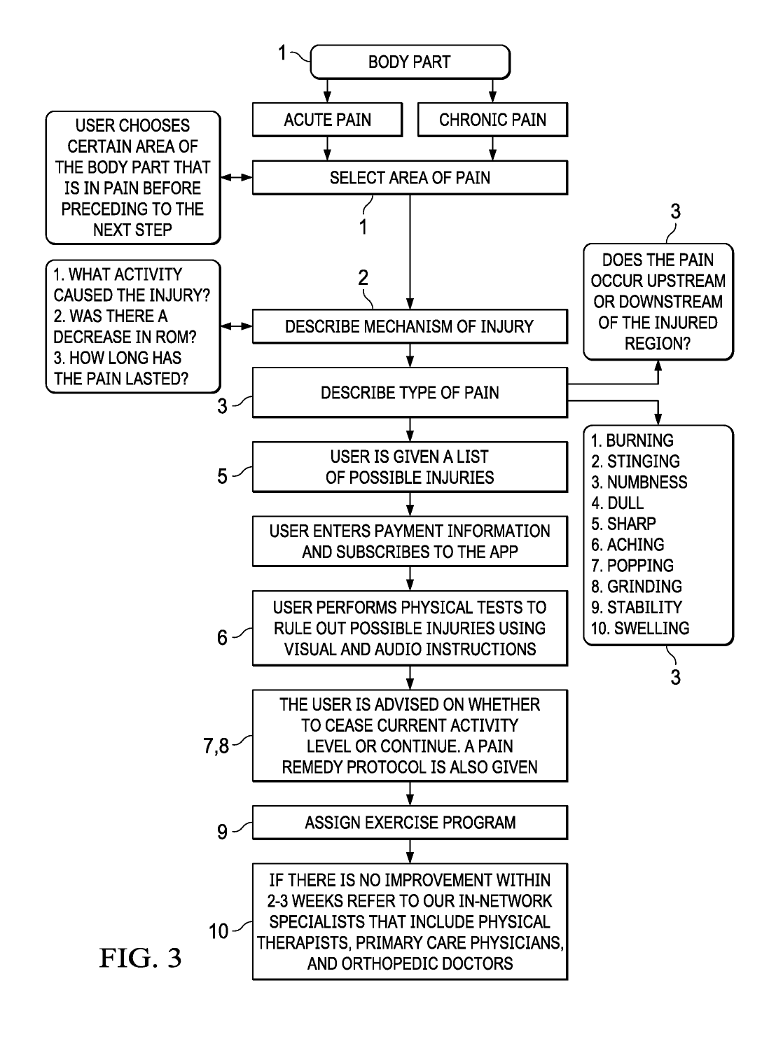

FIG. 3 shows an alternative injury flowchart and diagnosis/treatment with RRA. The user would classify their pain 3 as either acute or chronic and then the user would be prompted to select the injured body part 1 from a list provided by RRA. The user would then indicate the Mechanism of Injury 2 (i.e. what caused the injury, impact on range of motion, and how long the pain has affected the user). Thereafter the user would indicate the type of pain 3 the injury is causing. The user inputs will be processed through RRA and evaluated by the application's parameters to develop and provide a list of possible injuries 5 to the user. The user then has the option of submitting payment information and subscribing to RRA for further diagnosis and treatment. If the user decides to continue with RRA, then the user will perform, the prescribed physical tests 7 to rule out specific injuries. As input the user can answer RRA prompted questions and/or submit video or photographs of the user performing the physical tests to determine severity of injury and range of movement. Based on the RRA analysis of the physical test results, RRA will advise the user 8 to cease the current activity level or continue the current activity level. The user will also receive a pain remedy protocol, in which a user receives an exercise program 9 to repair and heal the identified injury. The treatment may be delivered by video, photographs, written description or through teleconference with the medical professional. If there is no improvement after a few weeks, RRA will offer the user referrals to medical professionals 8 such as physical therapists, primary care physicians, orthopedic doctors, etc. If there is marked improvement, the user will receive an exercise program to facilitate the continued healing process and strengthen the injured area to hopefully prevent future re-injury 11.

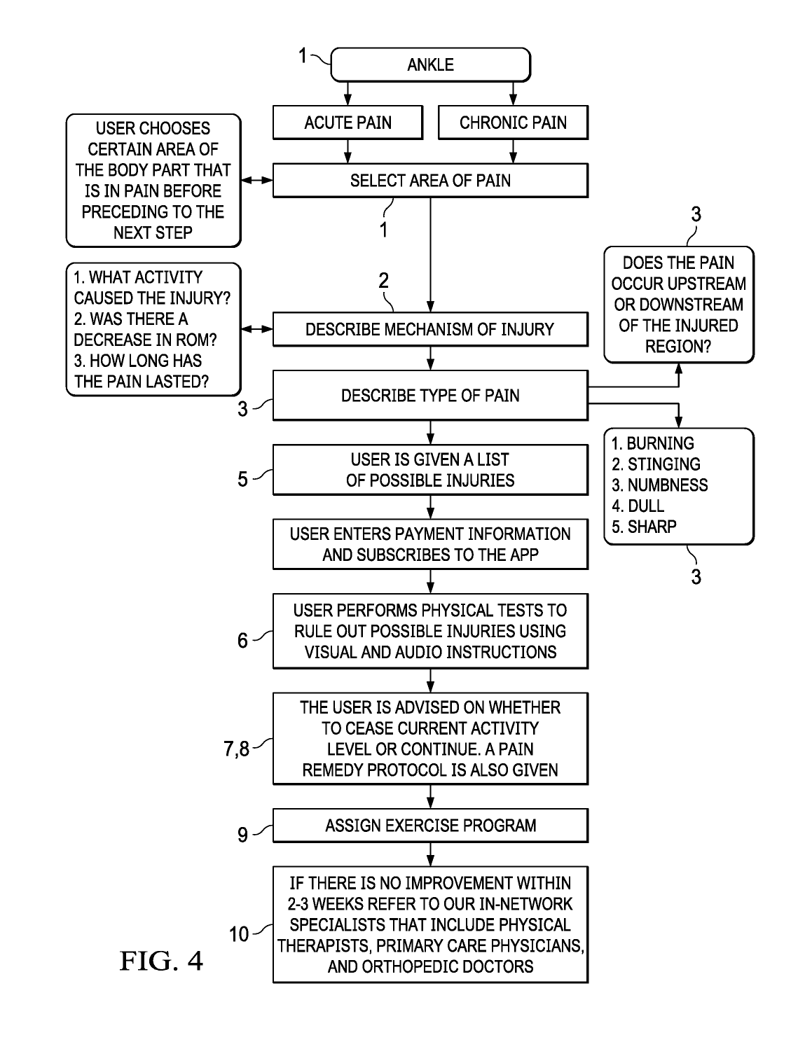

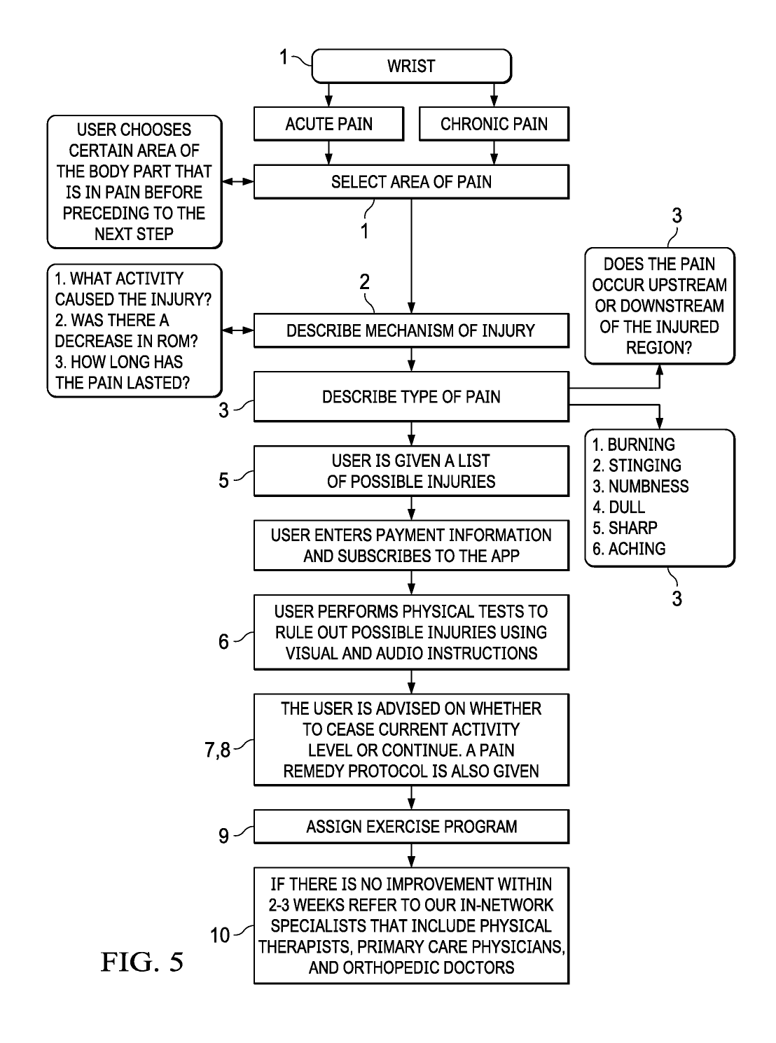

FIG. 4 shows the sample flowchart for diagnosis and treatment of an ankle injury. FIG. 5 shows the sample flowchart for diagnosis and treatment of a wrist injury.

It may be advantageous to set forth definitions of certain words and phrases used in this patent document. The term “couple” and its derivatives refer to any direct or indirect communication between two or more elements, whether or not those elements are in physical contact with one another. The terms “include” and “comprise,” as well as derivatives thereof, mean inclusion without limitation. The term “or” is inclusive, meaning and/or. The phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like.

While this disclosure has described certain embodiments and generally associated methods, alterations and permutations of these embodiments and methods will be apparent to those skilled in the art. Accordingly, the above description of example embodiments does not define or constrain this disclosure. Other changes, substitutions, and alterations are also possible without departing from the spirit and scope of this disclosure and the following claims.

### SUMMARY

Embodiments of the present disclosure, the Rehab Ready Application (RRA), generally provide an application that will allow a user to diagnose an injury that may have been caused by work automobile injury, exercise or other athletic ventures such as running, lifting weights, jumping, swimming, etc. and then identity specific exercises and treatment for various aches and pains due to those injuries. After a user experiences an injury to the shoulder, knee, ankle or other body part, the user can log into RRA, view an image of an anatomically correct human body, and select or click on a specific area of the body image within the app that corresponds to the part of the user's body 1 where the user is currently experiencing pain or other unpleasant symptoms. RRA prompts users to enter their injury symptoms and not be influenced by loads of irrelevant information. The RRA user cannot advance to the next section until they complete the present task or set of questions, allowing the RRA to methodically and scientifically develop an accurate assessment of the user's current injury based on user input and medical experience and knowledge of the human body. After completing the requested input an RRA user will be educated on the injury and their body, why the injury occurred, how to address or repair the injury, the timeline for recovery of the injury and how to prevent that particular injury in the future.

Initially the RRA will ask and determine if the pain is acute or chronic. 3 Acute pain may be caused by a traumatic event, lasts less than three month and the pain ceases once the injury heals. Chronic paid last more than three months, may not have a clear cause and acute injuries can become chronic over time. The RRA will ask the user to identity the area of pain 1 for a particular region of the body. The RRA will also ask the user to describe the mechanism of pain or mechanism of injury 2 (what activity caused the injury, was there an increase/decrease in range of motion, is the pain upstream or downstream of the injury, and how long has the pain lasted?) and how injury occurred (ex: pull up, deadlifting, muscle ups, squats, supinated grip pulling, rope climb, farmer's carry, etc.) and type of pain (burning, stinging, numbness, dull, sharp, aching, popping, grinding, loss of stability, swelling bruising, anatomical deformity, etc.). Based on the analysis of these factors (area of pain, mechanism of pain and type of pain), RRA will provide the user with a list of possible injuries. The user will perform physical tests to rule possible injuries using visual and audio communication with RRA. Upon completion of the questionnaire and physical tests, the user will provide payment information. The user will also receive suggestions of treatments to remedy the pain 7 (rest, ice, compression, elevation, NSAIDs, massage, heat, e-stem etc.) 9. If RRA determines that the injury is too severe to be self-treated by the patient, RRA will advise the user to report to the emergency room for immediate care. If the patient is to continue with RRA, the user will be asked to provide information on their activity level and effectiveness of the suggested pain remedy 10. RRA will analyze these entries and recommend a future exercise program. If improvement remains elusive, the user will be directed to an RRA in-network specialist (ex: physical therapists, primary care physicians, orthopedic doctors, etc.)

Once a user has recovered from the injury, either through self-treatment 9 with the assistance of RRA or in-person treatment from a RRA network provider, the user can return to RRA for guidance on strengthening and stability programs to prevent re-injury or aggravation of old injuries 11. RRA will maintain the user's history of injuries, treatments and medical providers within RRA for future reference. RRA provides specialized treatment options specifically tailored to the unique needs of individual users. Based on a user's responses to the RRA inquiries, a layman without medical training, can receive the benefit of the education and experience of a medical professional in the diagnosis and treatment of injury or ailments, from the comfort of home, through video conferences or through in-person appointments in case of severe injuries. For the most severe injuries requiring in person treatments, follow-up treatments and appointments can be conducted remotely through RRA.

For each area of the body selected by the user, a series of questions will be presented to the user to aid in the diagnosis of the user's ailments 5. The questions may be related to pain level 4, range of movement or type 3 and severity of pain 4. Depending on the responses from the user, the RRA will provide a general diagnosis 5 such as sprain, strain, muscle imbalance, swelling, stillness, etc. For each particular diagnosis, the RRA will suggest a set of exercises 7 with videos and photos for demonstration as well as written directions and audio recordings describing the treatment for the diagnosed ailment(s). The RRA self-treatment plan 9 will include information of how often to perform the exercises, load or weight for each repetition, and a progressive timeline. The user will also have the option of recording tasks completed and rehabilitation progress 10 within RRA. Thereafter, RRA medical professionals may also view the user's progress 10, as entered by the user in RRA, and suggest modifications to the initial rehabilitation program or other feedback, if necessary and requested by the user.

In cases where RRA determines that the user's injuries are too severe for mobile diagnosis and self-treatment 12. RRA directs the user to provider options from the RRA network of affiliated medical professionals. If RRA determines that an injury is an emergency, the user will be directed to an emergency room 12, primary care physician or other medical facility for immediate treatment.

The RRA network includes different practitioners and medical professionals with the expertise to treat the ailments and conditions of the RRA users. Any provider listed in the RRA network will be prescreened to ensure proper qualifications and experience. RRA is comprised of peer reviewed data, professional opinions based on years of training and experience treating the human body and body mechanics. The experts advising users of RRA are knowledgeable with respect to exercise, movement, physiology as it pertains to healing, rehabilitation, and coaching. The user can choose between a video call (i.e. telenet or telemedicine) evaluation 8 with a network practitioner or scheduling an appointment for an in-person evaluation at the facility of the practitioner.

Other technical features may be readily apparent to those skilled in the art from the included figures and descriptions.

What is claimed is:

1. A system for remote physical therapy comprising:
Requesting diagnostic data from a user;
Analyzing said diagnostic data;
Comparing said diagnostic data to common symptoms of known injuries;
Categorizing said diagnostic data to determine a course of treatment;
Recommending said course of treatment to said user;
Monitoring said user as said course of treatment is performed by said user;
Determining if said user needs more advanced exercises in order to reach desired rehabilitation outcome;
Determining if said user requires additional treatment due to severity of said injury; and
Recommending a medical professional to treat said user; and
Facilitating communication between said user and said medical professional.

2. The system of claim 1, wherein said diagnostic data is selected from the group consisting of area of pain, type of pain, and mechanism of injury.

3. The system of claim 1, wherein said user submits additional diagnostic data into said system through medium selected from the group consisting of: pictures, video, written description and oral description.

4. The system of claim 1, wherein said system directs said user to emergency care based on severity of said injury.

5. A platform for remote rehabilitation comprising:
requesting diagnostic data from a user;
providing suggestions of possible injuries to said user based on analysis of said diagnostic data;
requesting payment information from said user to access treatment options;
providing said treatment options to said user to further determine the source and severity of injury wherein said treatment options are selected from the group consisting of video instructions, audio instructions, visual instruction, and written instructions; wherein said user submits treatment performance results;
providing a pain remedy protocol to said user based on said user's treatment performance results;
providing an exercise program to said user based on analysis of said user's treatment performance results;

6. The platform of claim 5, wherein said platform recommends future treatment for said user based on said user's results from said exercise program.

7. The platform of claim 5, wherein said platform provides analysis of the cause of the injury, treatment of said injury, and preventative measures for said injury.

8. The platform of claim 5, wherein said platform retains user diagnostic data, treatment options, treatment performance results, and exercise programs for future access by said user.

9. The platform of claim 5, wherein said platform recommends future treatment for said user based on said user's results from said exercise program.

10. The platform of claim 9, wherein said platform provides analysis of the cause of the injury, treatment of said injury, and preventative measures for said injury.

11. The platform of claim 10, wherein said platform retains user diagnostic data, treatment options, treatment performance results, and exercise programs for future access by said user.

12. A computer application for diagnosis and treatment of a user comprising:
A database of injury mechanism;
A database of body parts;
A database of levels of pain;
A database of types of pain;
A database of types of injury;
A database of ranges of mobility;
A database of treatments
A database of exercises; and
A database of affiliated medical professionals for user referrals.

13. The computer application of claim 12 wherein said user provides answers to a series of questions, wherein said answers are compared to a series of databases within said computer application, wherein a course of treatment for said user's injury is determined.

14. The computer application of claim 12, wherein said application recommends future treatment for said user based on said user's results from said exercise program.

15. The computer application of claim 12, wherein said application provides analysis of the cause of the injury, treatment of said injury, and preventative measures for said injury.

16. The computer application of claim 12, wherein said application retains user diagnostic data, treatment options, treatment performance results, and exercise programs for future access by said user.
